(19) United States
(12) Patent Application Publication
(10) Pub. No.: US 2003/0236472 A1
(43) Pub. Date: Dec. 25, 2003

(54) Systems and methods for moving anatomical elements

(76) Inventors: James Van Hoeck, Cordova, TN (US); Bradley J. Coates, Rossville, TN (US); Jeffrey W. Poyner, Atoka, TN (US); Kevin T. Foley, Germantown, TN (US)

Correspondence Address: Woodard, Emhardt, Naughton,, Moriarty and McNett, Bank One Center/Tower, 111 Monument Circle, Suite 3700, Indianapolis, IN, 46204-5137, US

(21) Appl. No.: 10/260,891

(22) Filed: Sep. 30, 2002

Related U.S. Application Data
(60) Provisional application No. 60/390,131, filed on Jun. 19, 2002.

(51) Int. Cl. A61B 5/103

(52) U.S. Cl. 600/587

(57) ABSTRACT

Systems and methods for measuring a separation characteristic of separated anatomical elements include a separator contactable with first and second anatomical elements. A sensor provides an indication of the separation characteristic of the anatomical elements. The measured separation characteristic can be compared with a desired or predetermined separation characteristic, and subsequent surgical procedures can be performed based on the measured separation characteristic.

### CROSS-REFERENCE TO RELATED APPLICATION

This application claims the benefit of the filing date of Provisional Application No. 60/390,131 filed on Jun. 19, 2002.

### BACKGROUND

Anatomical elements can be moved or separated in surgical procedures to increase the working space available to access the space between, around and/or through the anatomical elements. For example, anatomical elements can include tissues of the patient that require separation to access a location in the patient's body. In another example, anatomical elements can include adjacent bony portions that require separation for insertion of an implant or the like between the bony portions.

Under-separation of the anatomical elements may not provide the desired or maximum access to the space between, around and/or through the elements. Under-separation can also result in additional wear and/or stress on an implant positioned between the anatomical elements. Over-separation of the anatomical elements can result in cutting or tearing of the tissue connecting the anatomical elements. Other complications can result depending on the location of tissue separation, such as dysphasia in anterior cervical approaches. Over-separation can also hyperextend the space between the anatomical elements. For example, if the anatomical elements were adjacent bony portions, a loose implant fit in the space between the elements may result with over-separation.

Over and under-separation of anatomical elements can be a surgical result stemming from the surgeon having to use subjective judgements, tactile feedback, and visual observations of the anatomical elements to determine the separation employed on the anatomical elements during the surgical procedure. Furthermore, differing separations of the same anatomical elements may be desirable for different types of implants and procedures. The lack of objective information regarding the anatomical elements can prevent the surgeon from achieving an optimal separation result for the surgical procedure.

### SUMMARY

There is provided systems and methods for moving at least one anatomical element relative to another and providing objective feedback of one or more separation characteristics of the separated anatomical elements. The separation characteristic(s) can provide an objective indication to the surgeon of the condition of the anatomical elements, which can then be employed to obtain a surgical result.

According to one aspect, there is a system and method for moving anatomical elements having a feedback system that determines a separation characteristic from the anatomical elements.

According another aspect, there is a system and method for moving anatomical elements having a feedback system that determines a separation characteristic from the anatomical elements. The separation characteristic is compared to a desired separation characteristic. When the desired separation characteristic is obtained, a construct is positioned in the space between or adjacent the anatomical elements.

According another aspect, there is a system and method for moving anatomical elements having a feedback system that determines a separation characteristic from the anatomical elements. The separation characteristic is compared to a threshold separation characteristic. When a threshold characteristic is obtained, further surgical steps can be undertaken. For example, a device can be inserted in the space between the anatomical elements, or engaged to the anatomical elements. In another example, steps can be taken to bring the separation characteristic below the threshold by, for example, moving the anatomical elements toward one another or increasing an incision size.

In one aspect, the system includes a first member having a feedback system associated therewith that is contactable with a first anatomical element. The feedback system determines a separation characteristic of the first anatomical element and a second anatomical element as the first anatomical element is moved relative the second anatomical element.

In one aspect, the system includes a first member having a feedback system associated therewith contactable with a first soft tissue element. The feedback system determines a separation characteristic of the first soft tissue element and a second soft tissue element as the first soft tissue element is moved relative to the second soft tissue element.

In another aspect, a surgical tool for moving soft tissue elements includes a first member contactable with a first soft tissue element. The first soft tissue element is spaced from a second soft tissue element. A feedback system associated with the surgical tool is operable to determine a separation characteristic of the first and second soft tissue elements.

In a further aspect, a system for moving soft tissue elements is provided. The system comprises a first member contactable with a first soft tissue element that is spaced from a second soft tissue element. A second member is contactable with the second soft tissue element. A feedback system associated with at least one of the first and second members is operable to determine a separation characteristic of the first and second soft tissue elements.

In one aspect, a system for moving a first soft tissue element and a second soft tissue element is provided. The system comprises a member contactable with the first soft tissue element and the second soft tissue element. The member maintains the first soft tissue element in a spaced apart relationship with respect to the second soft tissue element. A feedback system associated with the member determines a separation characteristic of the first and second soft tissue elements.

In another aspect, a system includes an instrument for moving a first soft tissue element and a second soft tissue element is provided. The instrument is contactable with the first soft tissue element and the second soft tissue element. A feedback system associated with the instrument is operable to determine a separation characteristic of the first soft tissue element and the second soft tissue element.

One aspect includes a system for moving first and second soft tissue elements. The system comprises a first member contactable with the first soft tissue element, and a second member contactable with the second soft tissue element. A third member, connectable with the first member and the second member, maintains the first soft tissue element in a spaced apart relationship with respect to the second soft tissue element. A feedback system is associated with the first member for determining a separation characteristic of the first and second soft tissue elements.

Another aspect includes a method for moving soft tissue elements. The method includes coupling a tissue separation system with soft tissue elements to be moved, moving the soft tissue elements, and determining a separation characteristic of the soft tissue elements.

In another aspect a tissue separation system is provided. The system includes a connector having first and second ends with a portion therebetween. A stationary arm is affixed to the first end and a movable arm is affixed to the second end. The movable arm is movable along the connector with respect to the stationary arm. A feedback system associated with the system is operable to determine a separation characteristic associated with the tissue during and/or after the movable arm and the stationary arm are moved relative to one another.

In a further aspect, a system for moving vertebral bodies is provided. The system comprises a connector having an engageable portion extending between opposite ends. A first member extends from one end of the connector and is connectable with a first vertebral body. A second member is movably engaged to the connector, and extends therefrom and is connectable with a second vertebral body. The second arm is movable along the connector with respect to the first arm. A system associated with at least one of the first arm and the second arm determines an actual separation characteristic, which is comparable to a desired or predetermined separation characteristic. Feedback is provided to the surgeon regarding the same. The actual separation characteristic can be adjusted based on the feedback to obtain a desired or predetermined separation characteristic.

In another aspect, a method for moving vertebral bodies includes coupling a vertebral body separator to the vertebral bodies to be moved, separating the vertebral bodies, and determining a separation characteristic of the vertebral bodies. The separation characteristic can be compared to desired or predetermined separation characteristics to determine if separation of the vertebral bodies is appropriate and/or should be increased or decreased for insertion of an interbody device or engagement of a plate or rod construct to the vertebrae.

In another aspect, a method for moving vertebral bodies includes coupling a vertebral body separator to the vertebral bodies to be moved, separating the vertebral bodies, and determining a separation characteristic of the vertebral bodies. If the determined separation characteristic corresponds to a desired separation characteristic, a construct is inserted in the space between or adjacent the vertebral bodies.

These and other aspects will also be apparent from the following description of the illustrated embodiments.

### BRIEF DESCRIPTION OF THE DRAWINGS

FIG. 1 is a block diagram of one embodiment of a system for separating anatomical elements and providing feedback regarding the same.

FIG. 2 is a block diagram of another embodiment system for separating anatomical elements and providing feedback regarding the same.

FIG. 3 is a block diagram of another embodiment system for separating anatomical elements and providing feedback regarding the same.

FIG. 4 is a block diagram of another embodiment system for separating anatomical elements and providing feedback regarding the same.

FIG. 5 is a block diagram of another embodiment system for separating anatomical elements and providing feedback regarding the same.

FIG. 6 is a block diagram of another embodiment system for separating anatomical elements and providing feedback regarding the same.

FIG. 7 is a chart for a method of separating soft tissue elements.

FIG. 8 is a chart for a method of separating vertebral elements.

FIG. 9 is a perspective view of one embodiment instrument for separating and providing feedback regarding the separation of anatomical elements.

FIG. 10 is an elevation view of another embodiment instrument for separating and providing feedback regarding the separation of anatomical elements.

FIG. 11 is a view of a portion of the instrument of FIG. 10 having an alternate embodiment feedback.

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is hereby intended. Any such alterations and further modifications in the illustrated devices, and any such further applications of the principles of the invention as illustrated herein are contemplated as would normally occur to one skilled in the art to which the invention relates.

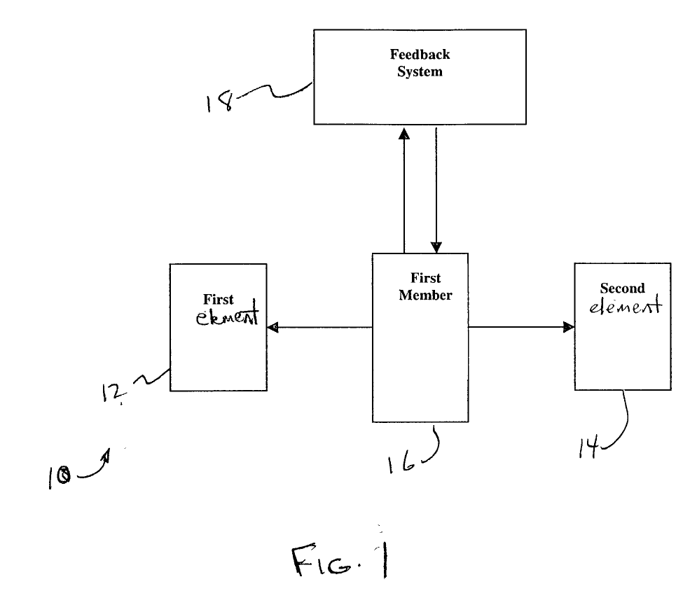

Referring to FIG. 1, there is shown a system 10 for moving at least a first anatomical element 12 relative to a second anatomical element 14. Anatomical elements 12 and 14 are located in a patient undergoing surgery, and require separation from one another or reducing the separation from one another as a result of a surgical procedure being performed on a patient. System 10 includes a member 16 contactable with first anatomical element 12. Member 16 can be manipulated by the surgeon to move first anatomical element 12 and second anatomical element 14 relative to one another. A feedback system 18 is associated with member 16 and provides immediate, real-time, and/or requested information to the surgeon regarding one or more separation characteristics associated with first and second anatomical elements 12, 14. Feedback system 18 can also provide the surgeon an indication when a desired or predetermined separation characteristic is obtained, and/or when certain threshold separation characteristics are obtained and/or approached.

Anatomical elements 12 and 14 may include any relatively soft or relatively hard organic tissue which can be moveable relative to one another either in their pre-surgical state or in an altered state provided during surgery. Suitable examples of anatomical elements include epithelial tissue, including the dermal layers of skin and body cavities; connective tissue, including loose connective tissue, dense connective tissue, adipose tissue, cartilage, blood, and bone; muscle tissue, including smooth muscle tissue, skeletal muscle tissue, and cardiac muscle tissue; adipose tissue; nervous tissue; vascular tissue, including blood vessels, veins, arteries, and capillaries; cartilage tissue, including hyaline cartilage tissue, elastic cartilage tissue, and fibrocartilage tissue; and bone, including spongy bone tissue, compact bone tissue, cortical bone tissue, and cancellous bone tissue.

Although the first and second anatomical elements can be organic tissues, they do not have to be the same type of organic tissue. It is contemplated, for example, first and second anatomical elements 12, 14 include opposing, interrupted tissue masses on opposite sides of a surgical incision. It is also contemplated first and second anatomical elements 12, 14 can be, for example, opposing endplates of adjacent vertebrae. It is further contemplated that first and second anatomical elements 12, 14 can be, for example, endplates of vertebrae separated by one or more in place or removed vertebrae. Also contemplated are first and second anatomical elements that are first and second vertebral bodies, which may be adjacent to one another or include one or more additional vertebrae therebetween. The first and second anatomical elements may each be either the anterior portion of a vertebra, or one or more of the posterior elements of a vertebra, such the spinous or transverse processes, the facets, pedicles or lamina, for example.

A member can include any device, mechanism or body capable of moving first anatomical element 12 and second anatomical element 14 relative to one another. A member may be placed in direct contact with the anatomical element, or contactable with the anatomical element with one or more fasteners, connecting elements or the like engaged to the anatomical element. Examples of members include tissue retractors, tissue spreaders, tissue forceps, vertebral body distractors, and disc space spreaders. Further examples of members include one or more retractor elements such as blades, arms, pins, scoops, valves, bodies, shafts, flanges, bodies, fingers, extensions, and tips or the like capable of contacting and moving one or more anatomical elements. The member can be configured to contact soft tissue anatomical elements. Additional examples of members include one or more distractor elements such as bodies, fingers, extensions, tips or the like that can be inserted along, adjacent, against or into and anatomical element. For example, the member can be configured to contact an endplate of a vertebral body, a connector engaged to a vertebrae, connected to an anterior or posterior element of a vertebral body, or otherwise configured to contact and move a hard or bony tissue element.

As used herein, feedback system includes any system associated with member 16 and capable of determining one or more separation characteristics associated with moving first anatomical element 12 relative to second anatomical element 14. The one or more separation characteristics may include a distance an anatomical element is moved, a force or load exerted on an anatomical element, a strain associated with moving anatomical elements 12, 14 relative to one another, relative angles between anatomical elements or angle of one of the anatomical elements, distance between the anatomical elements, pressure exerted on one or both anatomical elements, or any other quality capable of objectively representing or characterizing separation of the anatomical elements. The feedback system may receive an input representative of the separation characteristic from an input system and may further include one or more output systems such as a visual display, audio, haptic, graphical, voice or other communication/response to communicate the one or more separation characteristics to the surgeon. In one particular embodiment, for example, the feedback system includes indicia visible to the surgeon, such as an analog scale, digital readout, or the like.

Further, feedback systems may include a processing system having appropriate hardware and/or software for converting input data into representative objective separation characteristics. Additionally, the processing system may include appropriate hardware, software, and predetermined separation characteristic values for outputting an adjustment signal representing a change in position required to achieve a predetermined or desired separation characteristic. Suitable examples of feedback systems include any one or combination of a distance-measuring system, a force-measuring system, a strain-measuring system, a degree-measuring system, an angle-measuring system, and a pressure measuring system.

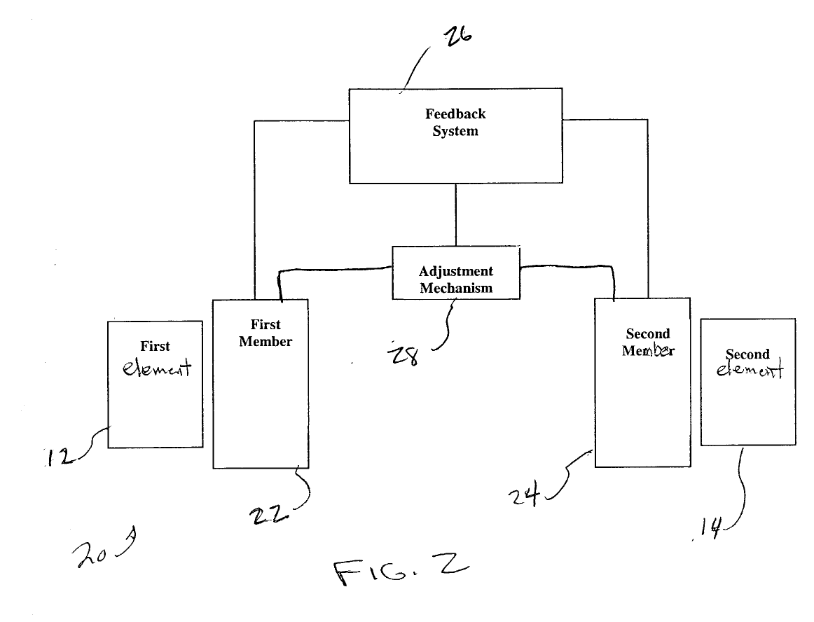

Referring to FIG. 2, there is shown another embodiment system 20 for moving at least first anatomical element 12 relative to second anatomical element 14. System 20 includes a first member 22 contactable with first anatomical element 12 and a second member 24 contactable with second anatomical element 14. First and second members 22, 24 can be manipulated by the surgeon to move first anatomical element 12 and second anatomical element 14 relative to one another. A feedback system 26 is associated with one or both of the first and second members 22, 24 to provide immediate, real-time, and/or requested objective information to the surgeon regarding one or more separation characteristics associated with the first and second anatomical elements 12, 14. Feedback system 26 can also provide the surgeon an indication when a desired or predetermined separation characteristic is obtained, when certain threshold separation characteristics are obtained, and/or as threshold separation characteristics are approached.

There is further provided an adjustment mechanism 28 associated with first and second members 22, 24 which is operable to move one or both of first and second members 22, 24 toward one another or away from one another. Adjustment mechanism 28 can be removably coupled to one or both of first and second members 22, 24, or permanently coupled to one or both of the first and second members 22, 24. Adjustment mechanism 28 can be associated with feedback system 26 so that the one or more separation characteristics can be determined in response to manipulation of the adjustment mechanism. Adjustment mechanism 28 can be provided with any of the separation systems discussed herein.

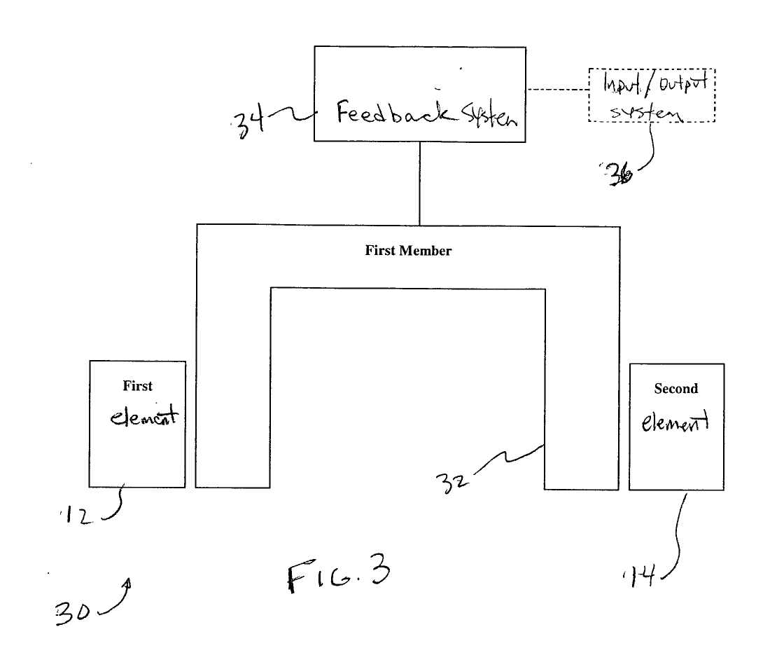

Referring to FIG. 3, there is shown another embodiment system 30 for moving at least first anatomical element 12 relative to second anatomical element 14. System 30 includes a member 32 contactable with each of the first anatomical element 12 and the second anatomical element 14. Member 32 can be manipulated by the surgeon to move at least one of the first anatomical element 12 and the second anatomical element 14 relative to the other. A feedback system 34 is associated member 32 to provide immediate, real-time information, and/or requested information to the surgeon, either directly or through input/output system 36, regarding one or more separation characteristics associated with the first and second anatomical elements 12, 14.

Feedback system 34 can also provide the surgeon, either directly or through input/output system 36, an indication when a desired or predetermined separation characteristic is obtained, and/or when certain threshold separation characteristics are obtained or approached. It is further contemplated that input/output system 36 can be used to provide information to feedback system 34 regarding pre-determined separation characteristic limits or desired separation characteristic results. These limits and/or results can be compared to one ore more actual separation characteristics determined by feedback system 34 and a signal provided to the surgeon indicative of the same through input/output system 36. Input/output system 36 can be provided with any of the separation systems discussed herein.

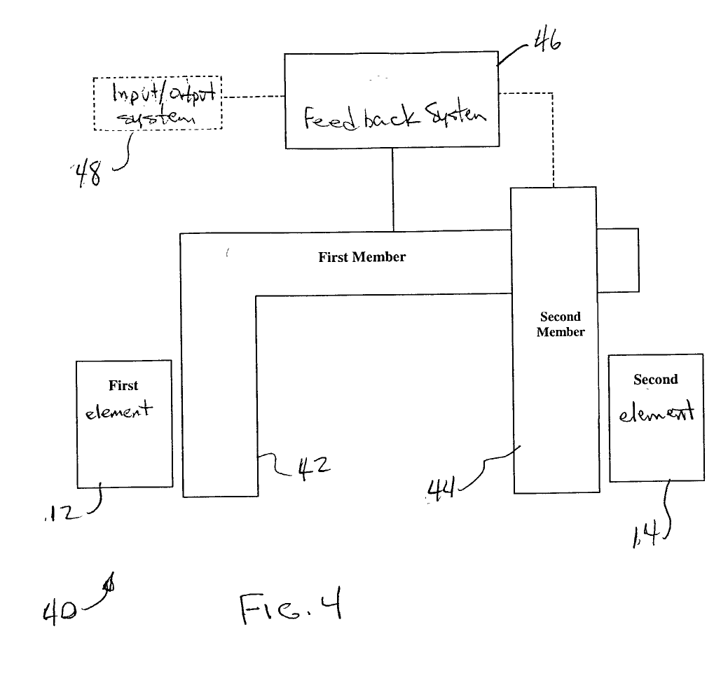

Referring to FIG. 4, there is shown another embodiment system 40 for moving at least first anatomical element 12 and second anatomical element 14. System 40 includes a first member 42 contactable with first anatomical element 12 and second member 44 contactable with second anatomical element 14. First and second members 42, 44 can be manipulated by the surgeon to move or separate first anatomical element 12 and second anatomical element 14 away from one another. Second member 44 is connected with a portion of first member 42 extending between first and second members 42, 44. Second member 44 is movable relative to first member 42 to separate first and second anatomical elements 12, 14.

A feedback system 46 can be associated with movable second member 44 to provide immediate or real-time information to the surgeon regarding one or more separation characteristics associated with moving the first and second anatomical elements 12, 14. Feedback system 46, either directly or through input/output system 48, can also provide the surgeon an indication when a desired or predetermined separation characteristic is obtained, and/or when certain threshold separation characteristics are obtained. It is further contemplated that input/output system 48 can be provided with information regarding pre-determined separation characteristic limits or desired separation characteristic results. These limits and/or results can be compared to actual separation characteristic determined by feedback system 46 and a signal provided to the surgeon indicative of the same through input/output system 48.

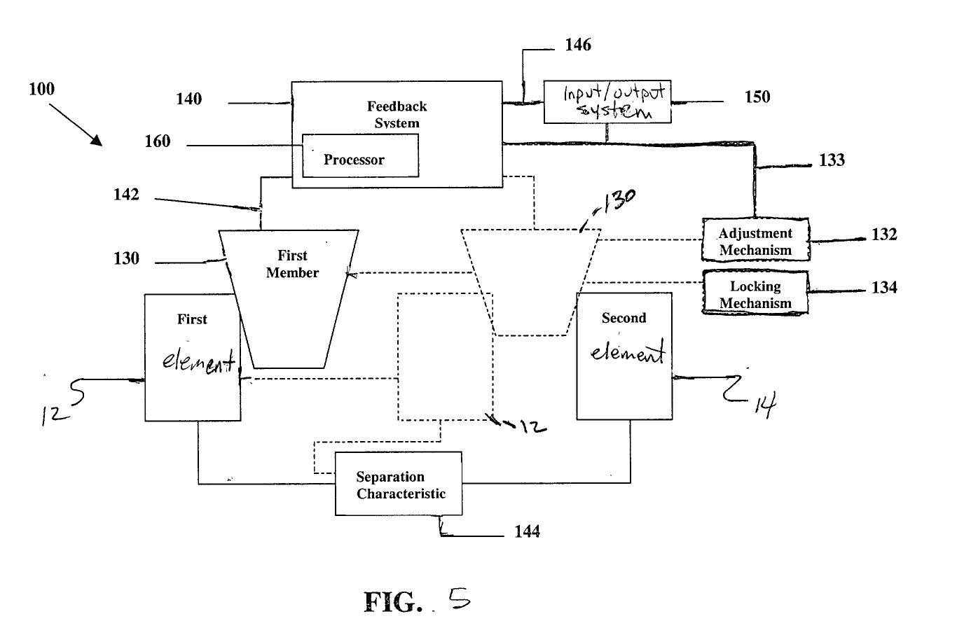

Referring to FIG. 5, another embodiment separation system 100 is shown. Separation system 100 includes a first member 130 that contacts first anatomical element 12, and creates or alters a spaced apart relationship between first anatomical element 12 and second anatomical element 14, from a first position, as indicated in dashed lines, and a second position, as indicated in solid lines for first member 130 and first anatomical element 12. Feedback system 140 is associated with first member 130, and receives feedback 142 representative of separation characteristic 144 of the first and second anatomical elements 12, 14. Further, feedback system 140 may include an output 146 that communicates one or more separation characteristics 144 to an attendant or surgeon. Thus, through first member 130 and feedback system 140, system 100 can measure separation characteristic 144 and thereby allow the attendant or surgeon to monitor, adjust and/or control the separation between first anatomical element 12 and second anatomical element 14 and/or to achieve a desired or predetermined separation characteristic.

An adjustment mechanism 132 can be associated with first member 130 and operable to further move apart or together first and second anatomical elements 12, 14. Feedback system 140 and/or processor 160 can be linked to provide a signal 133 to adjustment mechanism 132 in response to an input or measured separation characteristic. A locking mechanism 134 can lock adjustment mechanism 132 to maintain first and second anatomical elements 12, 14 in a separated or spaced position. Processor 160 can be provided with feedback system 140 and include hardware and/or software for converting inputs into representative separation characteristics. Additionally, the processing system may include appropriate hardware, software, and predetermined separation characteristic values for outputting an adjustment signal representing a change in position required to achieve a predetermined separation characteristic. Input/output system 150 can communicate via link 146 with feedback system 140 and/or processor 160 to provide input signals of desired or threshold separation characteristics and to receive output signals from feedback system 140 and/or processor 160.

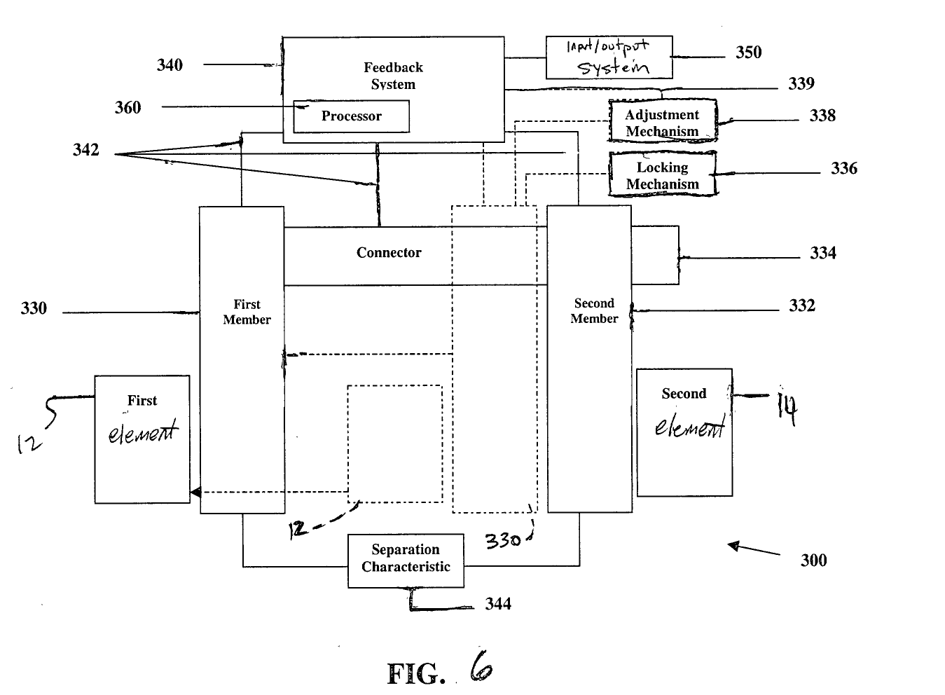

Referring to FIG. 6, another embodiment separation system 300 is shown. Separation system 300 includes a first member 330 that contacts first anatomical element 12 and a second member 332 that contacts second anatomical element 14. A connector 334 extends between and mechanically links first member 330 and second member 332. First and second members 330, 332 are moveable relative to one another to provide or alter a spaced apart relationship between first anatomical element 12 and second anatomical element 14 from a first position, as indicated in dashed lines, and a second position, as indicated in solid lines.

Feedback system 340 is associated with first and second members 330, 332 and receives feedback 342 representative of separation characteristic 344 of the first and second anatomical elements 12, 14. Further, feedback system 340 may include an input/output system 350 that communicates one or more separation characteristics 344 to an attendant or surgeon. Thus, through first member 330, second member 332 and feedback system 340, system 300 can measure separation characteristic 344 and thereby allow the attendant or surgeon to monitor, adjust and/or control the separation between first anatomical element 12 and second anatomical element 14, and/or to achieve a desired or predetermined separation characteristic.

An adjustment mechanism 338 can be associated with first member 330 and operable to further separate or release first and second anatomical elements 12, 14. Feedback system 340 and/or processor 360 can be linked to provide a signal 339 to adjustment mechanism 338 in response to an input or measured separation characteristic. A locking mechanism 336 can lock adjustment mechanism 338 to maintain first and second anatomical elements 12, 14 in a separated or spaced position. Processor 360 can be provided with feedback system 340 and include hardware and/or software for converting inputs into representative separation characteristics. Additionally, the processing system may include appropriate hardware, software, and predetermined separation characteristic values for outputting an adjustment signal representing a change in position required to achieve a predetermined separation characteristic. Input/output system 350 can communication via link with feedback system 340 and/or processor 360 to provide input signals of desired or threshold separation characteristics and to receive output signals from feedback system 340 and/or processor 360.

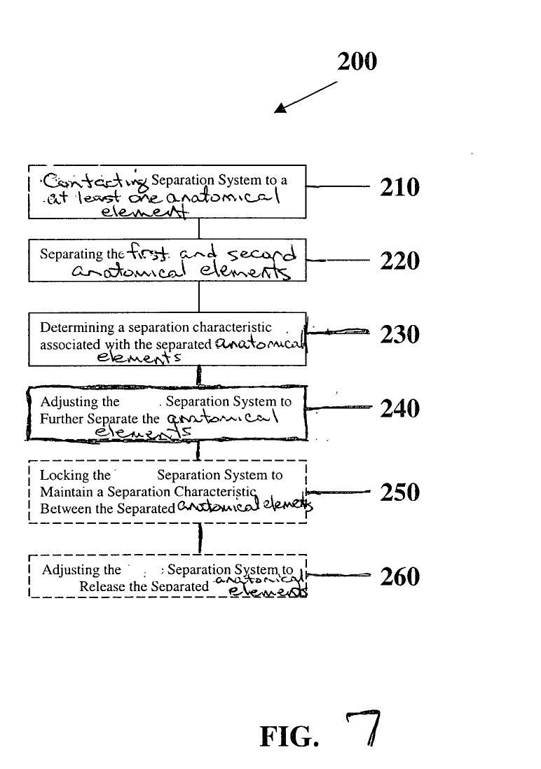

Referring to FIG. 7, a method 200 for moving first and second soft tissue anatomical elements is shown in block diagram form. At 210 the separation system is placed into contact with at least one of the two tissue elements be moved. For example, in methods employing separation system 100 of FIG. 5, first member 130 may be brought into contact with first tissue element, or brought into contact with an element or device in contact with first tissue element. Method 200 continues at 220 in which the two tissue elements are moved by moving at least one of the tissue elements away from the other tissue element. For example, in methods employing separation system 100 of FIG. 5, first member 130 may be moved from a first position to a second position, thereby creating or adjusting a space between first tissue element and the second tissue element.

At 230, a separation characteristic associated with moving the first and second tissue elements is determined. The separation characteristic can be determined during movement or after movement is complete. For example, in methods employing separation system 100, feedback system 140 may receive feedback 142 from first member 130 representative of a separation characteristic 144. Feedback system 140 may then provide, either directly or through an input/output system, an indication to the attendant or surgeon that is representative of separation characteristic 144.

Method 200 may further include at 240 adjusting the separation system and the spacing of the anatomical elements. For example, in methods employing separation system 100, first member 130 may be coupled to an adjustment mechanism 132, where adjustment mechanism 132 adjusts first member 130 to further separate or reduce separation of the tissue elements. The tissue elements can also be locked at 250 to maintain a separation characteristic associated with the anatomical elements. For example, with reference to separation system 100, first member 130 may further include locking mechanism 134 that is operable to lock first member 130 in a desired position for maintaining a separation characteristic 144 associated with the first and second tissue elements. It is further contemplated at 260 that the tissue can be released or moved toward one another by, for example, unlocking locking mechanism 134 or manipulating adjustment mechanism 132. The first and second tissue elements can be moved in closer proximity to one another and maintained in this closer proximity, or can be completely released to return toward their pre-separation position.

In one application of a method of moving soft tissue elements, an incision is made to access a surgical site on or near the spinal column. One specific application includes accessing the spinal column from an anterior approach to the cervical spine. Other applications contemplate other approaches, including posterior, postero-lateral, antero-lateral and lateral approaches to the spine, and accessing other regions of the spine, including the thoracic, lumbar and/or sacral portions of the spine. The first and second members of the separation instrument are inserted in the incision in contact with first and second tissue elements. The separation instrument is then manipulated to separate the tissue elements, and the feedback system measures one or more separation characteristics associated with the tissue elements.

A desired or threshold separation characteristic can be provided to or known to the surgeon prior to moving the tissue elements. For example, the desired separation characteristic can correspond to a maximum pressure or tension on the tissue elements determined through pre-operative planning or anatomical studies. The surgeon can maintain the actual separation characteristic measured during surgery at or below the desired or threshold separation characteristic. The feedback system can further be programmed to provide, either directly or through an input/output system, a warning, indication or comparison that the actual separation characteristic is approaching, the same as, or exceeding the desired or threshold separation characteristic. The surgeon can then select an appropriate course of action to maintain the actual separation characteristic within a desired limit or below a threshold separation characteristic. For example, the surgeon can stop tissue separation, reduce tissue separation, or increase the size of the incision. In this manner, the size of the incision can be optimized and made as small as possible in view of the tissue spacing required and the allowable separation characteristics.

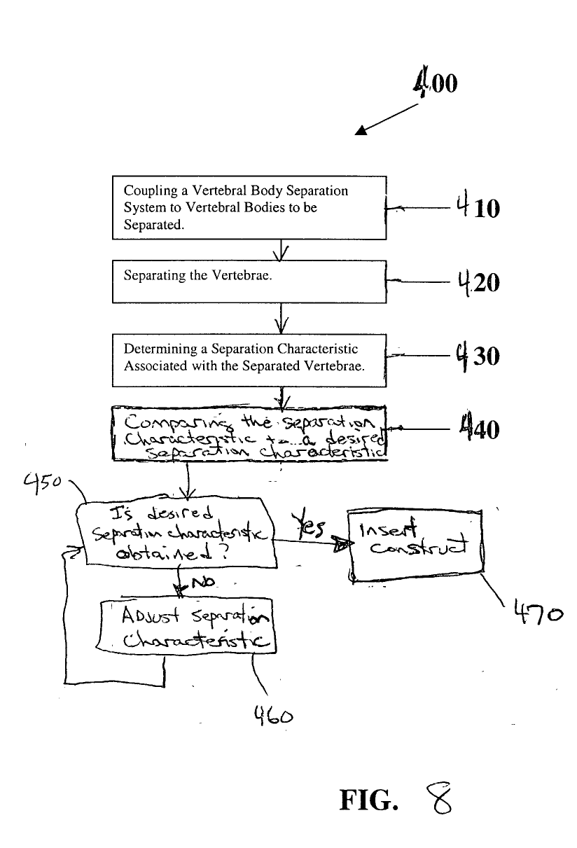

Referring to FIG. 8, a method 400 for moving first and second vertebral elements is shown in block diagram form. One application of method 400 is to increase a disc space height of a collapsed disc space between a pair of vertebrae. At 410 the separation system is coupled to at least one of the two vertebral elements be moved. Method 400 continues at 420 in which the two vertebral elements are separated by moving at least one of the vertebral elements relative to the other to increase a disc space height. The vertebrae can be locked in the separated position, or the separated position can be adjusted to increase the disc space height or decrease the disc space height with an adjustment mechanism.

At 430, a separation characteristic associated with moving the first and second vertebrae is determined. The separation characteristic can be determined during movement of the vertebrae or after movement is complete. For example, the load, pressure or force required to increase the disc space height can be determined during separation. The size of the space between the vertebral elements and/or the angulation between the vertebral elements can also be determined. The feedback system measures the separation characteristic and an indication of the same can be provided to the surgeon.

At 440, the determined separation characteristic is compared to a desired, predetermined or threshold separation characteristic, which can be developed through pre-operative planning, anatomical studies, and/or during surgery. For example, the predetermined or threshold separation characteristic can correspond to a maximum pressure or tension to be exerted by the members of the instrument on anatomical features of the vertebrae. The surgeon can maintain the actual separation characteristic measured during surgery to a level at or below the predetermined or threshold separation characteristic. The feedback system can further be programmed to provide, either directly or through an input/output system, a warning, indication or comparison that the actual separation characteristic is approaching, the same as, or exceeded the predetermined or threshold separation characteristic. The surgeon can then select an appropriate course of action to maintain or adjust the actual separation characteristic.

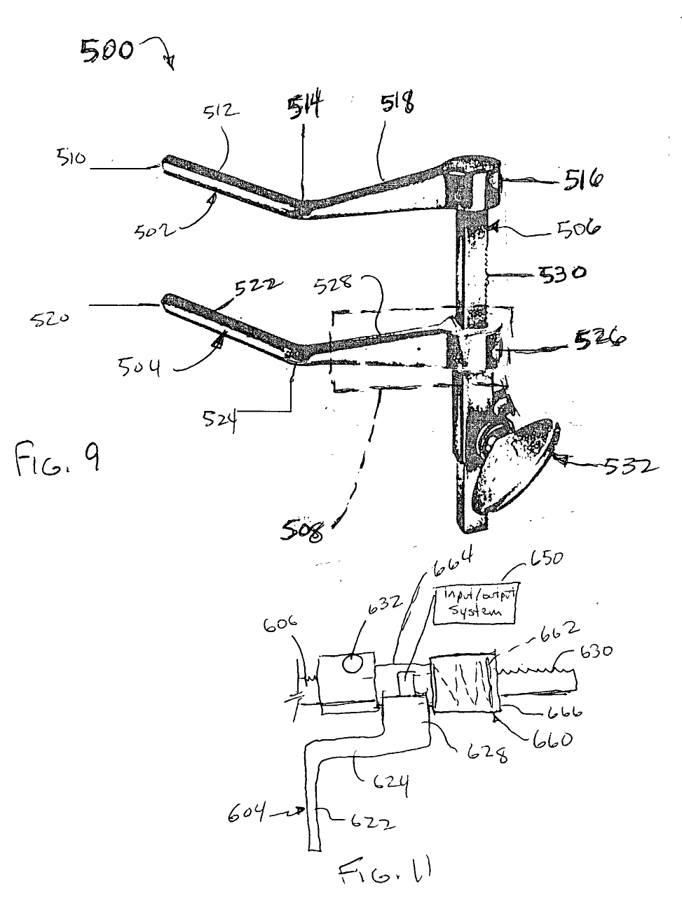

One example of a separation instrument is shown in FIG. 9. Separation instrument 500 includes a first member 502 and a second member 504. A connector 506 extends between first member 502 and second member 504. A feedback system 508 is operable to provide to the attendant or surgeon an indication of at least one separation characteristic of the anatomical elements in response to movement of at least one of the first and second members 502, 504 relative to the other. An adjustment mechanism 532 can be provided and manipulated by the attendant or surgeon to facilitate movement of first and second members 502, 504 relative to one another.

First member 502 includes a first end 510 and an opposite second end 516. Second end 516 is connected with connector 506. In the illustrated embodiment, second end 516 includes a ring extending about and immovably engaged to or formed with one end of connector 506. An extension 518 extends from second end 516 to an intermediate portion 514. A contacting portion 512 extends from intermediate portion 514 to first end 510. Second member 504 includes a first end 520 and an opposite second end 526. Second end 526 is connected with connector 506. In the illustrated embodiment, second end 526 includes a ring extending about and movably engaged to the body of connector 506. Accordingly, connector 506 and first member 502 can be moved relative second member 504. An extension 528 extends from second end 526 to an intermediate portion 524. A contacting portion 522 extends from intermediate portion 524 to first end 520.

Contacting portions 512, 522 can be, for example, a retractor element if instrument 500 were employed in a tissue retraction procedure, or a distractor element if separation instrument 500 were employed in a procedure moving bony portions. It is further contemplated that the entire length of contacting portions of first and second members 502, 504 can contact the anatomical element, only a portion of the length can contact the anatomical element, or the contacting portion can be engaged to a member or coupling element engaged to or in contact with the anatomical element. Contacting portions 512, 522 can be angled away from the remaining portions of instrument 500 so that instrument 500 can be maintained out of the way of the approach to the surgical site.

Connector 506 can be provided with engagement teeth 530 therealong for engagement with adjustment mechanism 532. Adjustment mechanism 532 is engaged or coupled with second member 504. In the illustrated embodiment, adjustment mechanism 532 is a thumbwheel coupled to a pinion member (not shown) having teeth that interdigitate with teeth 530 to move connector 506 and thus first member 502 relative to second member 504. Adjustment mechanism 532 can be manipulated to modify or adjust the separation characteristic to obtain a desired separation characteristic. Adjustment mechanism 532 may comprise manual thumbscrew, a deflection beam torque wrench, a spring ratchet, or similar mechanism. A locking mechanism can also be provided to prevent adjustment mechanism 532 and/or connector 506 from being moved so that a separation characteristic can be maintained during the surgical procedure.

Feedback system 508 may comprise a distance-measuring system, an angle measuring system, a force-measuring system, a change in force per change in distance measuring system, a pressure measuring system, and/or a strain measuring system. It is contemplated that feedback system 508 can be coupled to an output system to provide the surgeon an indication of the separation characteristic measured by feedback system 508. For example, a scale could be provided along or adjacent connector 506 to provide an indication of the separation characteristic. Feedback system 508 can also be coupled to other output systems, such as an oscilloscope, which can be operable to chart one or more of the separation characteristics.

In one embodiment, feedback system 508 measures the force applied to the anatomical elements versus the distance the anatomical elements are moved relative to one another. This separation characteristic is plotted along a chart, and when the applied force results in little or no change in distance between the anatomical elements, the increase in the separation force is stopped. The chart can provide a visual indication to stop separation of the anatomical elements since the distance component of the chart will not change as the distraction force increases.

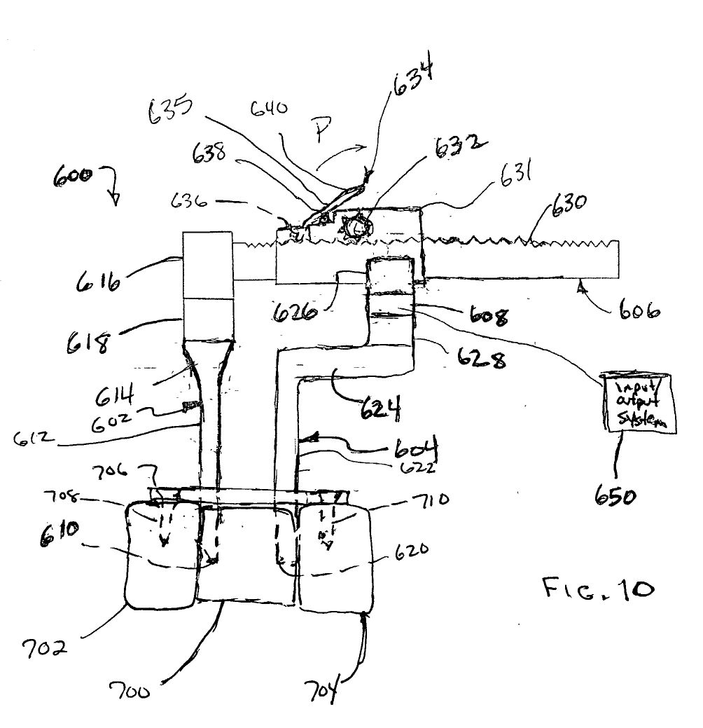

Another example of a separation instrument is shown in FIG. 10. Separation instrument 600 includes a first member 602 and a second member 604. A connector 606 extends between first member 602 and second member 604. A feedback system 608 is operable to provide to the attendant or surgeon an indication of at least one separation characteristic of the anatomical elements in response to movement of at least one of the first and second members 602, 604 relative to the other. An adjustment mechanism 632 can be provided and manipulated by the attendant or surgeon to facilitate movement of first and second members 602, 604 relative to one another.

First member 602 includes a first end 610 and an opposite second end 616. Second end 616 is connected with connector 606. In the illustrated embodiment, second end 616 includes a ring extending about and immovably engaged to or formed with one end of connector 606. An extension 618 extends from second end 616 to an intermediate tapered portion 614. A contacting portion 612 extends from intermediate portion 614 to first end 610. Contacting portion 612 can be angled relative to extension 618 to position the remaining portion of the instrument away from the approach to the surgical site.

Second member 604 includes a first end 620 and an opposite second end 626. Second end 626 is connected with connector 606. In the illustrated embodiment, second end 626 includes a ring-like element extending about and movably engaged to the body of connector 606. Accordingly, second member 604 can be moved relative first member 602. An extension 628 extends from second end 626 to an intermediate tapered portion 624. A contacting portion 622 extends from intermediate tapered portion 624 to first end 620. Intermediate portion 624 can extend along connector 606 to position contacting portion 622 adjacent contacting portion 612. Contacting portion 622 can be angled relative to extension 628 to position the remaining portion of the instrument away from the approach to the surgical site.

Contacting portions 612, 622 can be, for example, a retractor element if instrument 600 were employed in a tissue separation procedure, or a distractor element if separation instrument 600 were employed in a procedure moving bony portions. It is further contemplated that the entire length of contacting portions of first and second members 602, 604 can contact the anatomical element, only a portion of the length can contact the anatomical element, or the contacting portion can be engaged to a member or coupling element engaged to or in contact with the anatomical element.

Connector 606 can be provided with engagement teeth 630 therealong for engagement with adjustment mechanism 632. Adjustment mechanism 632 can be positioned in housing 631 extending about second member 604. In the illustrated embodiment, adjustment mechanism 632 is a pinion wheel that is rotatable along and in interdigitating engagement with one or more of the teeth 630 to move second member 604 along connector 606. Adjustment mechanism 632 can be manipulated with a thumbwheel or tool engagement device (not shown) to modify or adjust first and second members 602, 604 to obtain a desired or adjusted separation characteristic.

A locking mechanism 634 can also be provided to prevent adjustment mechanism 632 and/or second member 604 from being moved relative to connector 606. In the locked condition the separation characteristic can be maintained during the surgical procedure. In the illustrated embodiment, locking mechanism 634 includes a lever 635 having an end 636 selectively engageable and biased into engagement with teeth 630 of connector 606. Lever 635 is pivotally coupled at 638 to housing 631 adjacent second end 626 of second member 604. End 636 can be rotated in the direction indicated by arrow P to move out of engagement with teeth 630 and allow adjustment mechanism 632 to be used to adjust the separation characteristic. Furthermore, when unlocked, first and second members 602, 604 can be moved toward one another to release the anatomical elements or reduce the separation between the anatomical elements. When the desired separation characteristic is obtained, lever 635 of locking mechanism 634 can be rotated or biased in the direction opposite arrow P to engage teeth 630.

Feedback system 608 may comprise a distance-measuring system, an angle measuring system, a force-measuring system, a change in force per change in distance measuring system, a pressure measuring system, and/or a strain measuring system. It is contemplated that feedback system 608 can be coupled to an output system 650 to provide the surgeon an indication of the separation characteristic measured by feedback system 608. For example, a scale could be provided along or adjacent connector 606 to provide an indication of the separation characteristic. Feedback system 608 can also be coupled to other output systems, such as an oscilloscope, which can be operable to chart, plot, or provide a display indicative of one or more of the separation characteristics.

In the illustrated embodiment, feedback mechanism 608 can be a strain gauge coupled to extension 628 of first member 604 adjacent connector 606. The strain gauge can be positioned to measure the shear forces in extension 628 created by the distraction forces exerted on first member 604. It is further contemplated that the strain gage could be alternatively or additionally positioned on extension 618 to measure the tensile bending forces and/or the compression bending forces. Feedback system 608 could additionally or alternatively be coupled to first member 602, coupled to adjustment mechanism 632, and/or connector 606.

In FIG. 11 there is shown an alternate embodiment feedback system 660 with separation instrument 600, it being understood that feedback mechanism 660 could be employed with the other separation instrument embodiments. Feedback mechanism 660 includes a housing 666 in which spring 662 is positioned. Spring 662 is coupled to or in communication with extension 628 via a coupling member 664, and is responsive to loads exerted on second member 604 to provide a signal indicative of a separation characteristic to input/output system 650.

Although instruments 500 and 600 are shown with gear and rack or ratcheting type connectors between the first and second members of the separation instruments, other means for moving first and second members, or for moving a member of the separation instrument, are also contemplated. For example, such means may include a scissors-type connector between the first and second members, one or more resilient hinges or pivot pins between first and second members, ratchet mechanisms, motor and drive shaft, or linkages. For instruments employing a single contacting member, the member may be coupled to a proximal portion with a scissors-type connector, one or more resilient hinges or pivot pins, a ratchet mechanism, motor and drive shaft, or linkage, for example.

In one example of a surgical technique employing the separation instrument of the present invention will be discussed with reference to FIG. 10. An interbody construct 700, such as an intradiscal fusion device or corpectomy device, is to be inserted in the disc space between adjacent vertebrae 702, 704. It has been found that the force required to distract the vertebrae 702, 704 is approximately linearly related to the compressive force exerted on the interbody fusion or corpectomy device 700 when the distraction force is released. In one specific example, the residual compressive force can be in the range from 5% to 15% of the applied distractive load. Accordingly, the surgeon can distract the vertebrae 702, 704 to achieve a desired separation characteristic which corresponds to a desired compressive force that will be exerted on the interbody construct 700 inserted between the vertebrae 702, 704 when the separation instrument is removed. The compression loading of the interbody construct 700 facilitates the fusion of the adjacent vertebrae and helps retain the interbody construct 700 in the space between the vertebrae 702, 704. Accordingly, the separation instrument can provide the surgeon an objective indication of the compression loading that will be applied to the inserted interbody construct 700, and distraction of the vertebra can be optimized and/or adjusted accordingly to achieve a desired separation characteristic.

In another example employing the separation instrument, a construct 700 in the form of an artificial disc device is to be inserted in the disc space between adjacent vertebrae 702, 704. It can be desirable to provide a separation characteristic for an artificial disc device that would differ for an interbody fusion device. For example, greater post-operative compression loading is desirable for an interbody fusion device to facilitate fusion and maintain implant positioning before and during fusion. Lesser post-operative compression loading is desirable for an artificial disc since fusion between the vertebrae is not sought, and reduced compression forces will reduce wear of the artificial disc components. The surgeon can obtain a separation characteristic corresponding to a distraction force applied to the adjacent vertebrae, which corresponds to a desired compressive force that will be exerted on the artificial disc when the separation instrument is released. Accordingly, the separation instrument can provide the surgeon an objective indication of the compression loading that will be applied to the artificial disc to be inserted, and distraction of the vertebra 702, 704 can be adjusted accordingly to achieve a desired separation characteristic prior to or after insertion of the artificial disc device.

According to the present invention, it is contemplated that an interbody construct 700 can be inserted in the disc space between adjacent vertebrae 702, 704. The distractive force applied to separate vertebral bodies can be used to predict compressive forces that will be post-operatively exerted on the interbody construct 700 when the distraction force is released. In addition, a compression load between the vertebrae 702, 704 can be applied to provide additional compression loading to the interbody construct 700. The separation characteristic between the vertebrae 702, 704 supplied by the pre-insertion distraction forces and/or applied post-construct insertion can be post-operatively maintained by securing an extra-vertebral construct 706 to the vertebrae to maintain the compression loading against the inserted interbody construct 700. The separation instrument of the present invention can be employed for any one or all of pre-insertion distraction, post-insertion compression and to determine separation characteristics associated therewith.

The measured separation characteristic can be compared to a desired separation characteristic, adjusted if necessary, and when acceptable, the extra-vertebral construct 706 can be secured to the adjacent vertebrae 702, 704 with bone engaging fasteners 708, 710, respectively, to maintain the desired compressive load on the interbody construct 700. For example, the pre-insertion distraction force indicated by the separation instrument may indicate that that the post-insertion compression loading of the interbody construct 700 will not be sufficient. Accordingly, the distraction force can be increased.

The distraction force may not be able to be increased due to, for example, a maximum force limitation or threshold. In such cases, and in other cases where desired or necessary after insertion of the interbody construct 700, a compressive load can be applied with the separation instrument. The separation instrument measures the compression loading and when a desired compression loading is obtained, the extravertebral construct 706 can be secured to the vertebrae 702, 704 to maintain the compression loading. Engagement of the extra-vertebral construct 706 to the adjacent vertebrae can prevent or resist subsidence of the interbody construct 700 into the vertebral endplates, thus enabling higher compression loading of the interbody construct 700 to facilitate, for example, bony incorporation or anchorage of the interbody construct 700 to the vertebral endplates. It is contemplated that the extra-vertebral construct 706 can be a plating system, rod system, tethering system or other suitable system for engaging vertebrae 702, 704 extra-vertebrally. It is further contemplated the interbody construct 700 can be a fusion device, spacer, or artificial disc, for example.

While the invention has been illustrated and described in detail in the drawings and foregoing description, the same is to be considered illustrative and not restrictive in character. All changes and modifications that come within the spirit of the invention are desired to be protected.

What is claimed is:

1. A method positioning a spinal construct in a patient, comprising:
providing a separation instrument having a feedback system operable to provide objective data regarding at least one separation characteristic of anatomical elements;
making an incision in the patient to provide first and second anatomical elements;
positioning the separation instrument in contact with at least one of the first and second anatomical elements;
separating the first and second anatomical elements with the separation instrument;
objectively measuring the at least one separation characteristic with the feedback system, wherein the at least one separation characteristic includes a force applied to separate the first and second anatomical elements;
comparing the measured separation characteristic with a desired separation characteristic; and
positioning a construct in the space between the separated anatomical elements when the measured separation characteristic corresponds to the desired separation characteristic.

2. The method of claim 1, further comprising:
comparing the measured separation characteristic to a threshold separation characteristic; and
moving the first and second anatomical elements relative to one another obtain a second measured separation characteristic below the threshold characteristic.

3. The method of claim 1, wherein positioning the construct in the space includes positioning the construct through the space between the separated first and second anatomical elements.

4. The method of claim 3, wherein the first and second anatomical elements include soft tissue elements.

5. The method of claim 3, wherein the first and second anatomical elements include bony tissue elements.

6. The method of claim 5, wherein the desired separation characteristic corresponds to a compressive force applied to the construct when the construct is engaged to the first and second bony tissue elements and the separation instrument is released from at least one of the first and second bony tissue elements.

7. The method of claim 6, further comprising engaging the construct to the first and second bony tissue elements when the measured separation characteristic substantially corresponds to the desired separation characteristic.

8. The method of claim 7, wherein the construct is an implant positioned in an intervertebral space between the first and second bony tissue elements.

9. The method of claim 7, further comprising engaging a plate to the first and second bony tissue elements to maintain the compressive force.

10. The method of claim 7, wherein the construct is a plate extending across an intervertebral space defined by the first and second bony tissue elements.

11. A method for separating first and second vertebral bodies, comprising:
positioning a separation instrument in contact with the first vertebral body and the second vertebral body, the separation instrument including a feedback system associated therewith to provide objective data regarding at least one separation characteristic of the first and second vertebral bodies;
separating the first and second vertebral bodies;
determining a separation characteristic associated with the separated first and second vertebral bodies, wherein the separation characteristic includes an objective indication of a force applied to separate the first and second vertebral bodies; determining if the separation characteristic substantially corresponds to a predetermined separation characteristic; and
adjusting the separation characteristic based on the determination.

12. The method of claim 11, further comprising engaging a construct to the first and second vertebrae when the separation characteristic substantially corresponds to the predetermined separation characteristic.

13. The method of claim 12, wherein engaging the construct includes positioning an implant positioned in the intervertebral space between the first and second vertebral bodies.

14. The method of claim 13, wherein engaging the construct further includes engaging a plate to the first and second vertebral bodies.

15. The method of claim 12, wherein engaging the construct further includes engaging a plate to the first and second vertebral bodies.

16. The method of claim 11, wherein the force is indicated as a pressure.

17. The method of claim 11, wherein the force is indicated as a change in force per change in separation distance.

18. The method of claim 11, wherein the predetermined separation characteristic corresponds to a residual compressive force applied by the first and second vertebral bodies to a construct engaged between the first and second vertebral bodies when the separation instrument is released from at least one of the first and second vertebral bodies.

19. The method of claim 18, further comprising engaging the construct to the first and second vertebral bodies when the separation characteristic substantially corresponds to the desired separation characteristic.

20. The method of claim 11, wherein adjusting the separation characteristic further comprises applying a compressive load to the vertebral bodies after engaging a construct in the intervertebral space between the first and second vertebral bodies to provide a desired post-operative compressive load on the construct.

21. The method of claim 20, wherein the implant is an interbody fusion device.

22. The method of claim 20, further comprising engaging a plate to the first and second vertebral bodies to post-operatively maintain the compressive load.

23. The method of claim 20, wherein the implant is an artificial disc.

24. The method of claim 20, wherein the construct is a plate engaged to the first and second vertebral bodies.

25. The method of claim 19, wherein the compressive load is applied with the separation instrument to provide an objective indication of the compressive load.

26. The method of claim 11, wherein adjusting the separation characteristic includes reducing the force applied to separate the first and second vertebral bodies.

27. A system for moving first and second soft tissue elements relative to one another, comprising:
a connector having an engageable portion extending between opposite ends;
a first member extending from said connector contactable with a first soft tissue element;
a second member extending from said connector and contactable with a second soft tissue element spaced from the first soft tissue element, said second member being movable relative to said first member; and
a system associated with at least one of said first member and said second member operable to determine an actual separation characteristic associated with separation of the first and second soft tissue elements and provide objective feedback regarding the same, wherein said first member and said second member are movable relative to one another to adjust said actual separation characteristic in accordance with the objective feedback to obtain a desired separation characteristic.

28. The system of claim 27, wherein the objective feedback includes at least one from the group consisting of: a separation distance between the first and second soft tissue elements; a force applied to separate the first and second soft tissue elements; and a change in force per change in separation distance between the first and second soft tissue elements.

29. The system of claim 27, further comprising an adjustment mechanism coupled between said connector and said second member operable to move said second member relative to said first member.

30. The system of claim 29, further comprising a locking mechanism for securing said first member and said second member in a position relative to one another.

31. The system of claim 30, wherein said locking mechanism includes a spring loaded lever selectively engageable with a ratcheting surface provided along said connecting member.

32. The system of claim 31, wherein said adjustment mechanism includes a wheel having a number of teeth thereabout engageable with said ratcheting surface.

33. The system of claim 27, wherein said system includes a sensor associated with at least one of said first member and said second member.

34. The system of claim 33, wherein said sensor includes at least one selected from the group consisting of: a strain gauge, a spring; and a pressure sensor.

35. The system of claim 33, wherein:
said second member includes a housing positioned about said connector;
said sensor includes a spring in said housing coupled with a tissue contacting portion of said second member, said spring being responsive to a load on said tissue contacting portion to provide an indication corresponding to said actual separation characteristic.
